(19) United States
(12) Patent Application Publication
(10) Pub. No.: US 2003/0022906 A1
(43) Pub. Date: Jan. 30, 2003

(54) Use of pde v inhibitors

(76) Inventors: Marian Brandle, Rottenburg (DE); Thomas Ehring, Remscheid (DE); Claudia Wilm, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/220,416

(22) PCT Filed: Feb. 13, 2001
(86) PCT No.: PCT/EP01/01557

(30) Foreign Application Priority Data
Mar. 3, 2000 (DE) 10010612.9

(51) Int. Cl. A61K 31/519

(52) U.S. Cl. 514/267

(57) ABSTRACT

The invention relates to the use of a highly penis-specific PDE V inhibitor, or a physiologically acceptable salt or solvate thereof, for the production of a medicament for the treatment of erectile dysfunction in males, without the previous circulatory side effects caused by PDE V inhibitors, in particular, with concomitant administration of vasodilators, whose mode of action is by means of the NO/cGMP system.

The invention relates to the use of a highly penis-specific PDE V inhibitor, or a physiologically acceptable salt and/or solvate thereof, for the preparation of a medicament for the treatment of erectile dysfunction in men without circulatory side effects caused by PDE V inhibitors.

The use of PDE V inhibitors is described, for example, in WO 94/28902. Pyrimidine derivatives are known, for example, from EP 201 188 and WO 93/06104.

The use of PDE (phosphodiesterase) V inhibitors for the treatment of erectile dysfunction is known, for example, from EP 702 555 and WO 99/55708.

The best-known representative of the PDE V inhibitors is sildenafil (Viagra®).

Side effects which are observed on treatment of erectile dysfunction by PDE V inhibitors include cardiovascular side effects, such as a reduction in blood pressure and a reflex increase in heart rate, in particular in the case of nitrate interactions.

Thus, the use of sildenafil is contraindicated during administration of nitrate.

The administration of nitrate increases haemodynamic side effects of non-penis-specific PDE V inhibitors.

Known nitrate compounds are, for example, nitroglycerine=Nitrolingual® or Isoket®.

The invention had the object of finding novel compounds having valuable properties, in particular those which can be used for the preparation of medicaments.

Surprisingly, it has been found that highly penis-specific PDE V inhibitors, or physiologically acceptable salts and/or solvates thereof, can be used for the treatment of erectile dysfunction in men without simultaneous circulatory side effects caused by PDE V inhibitors.

In particular, no simultaneous circulatory side effects of non-penis-specific PDE V inhibitors occur.

These side effects can occur, in particular, if vasodilators which act via the NO-cGMP system are administered simultaneously.

The invention therefore also relates to the use of a highly penis-specific PDE V inhibitor, or a physiologically acceptable salt and/or solvate thereof, for the preparation of a medicament for the treatment of erectile dysfunction in men without circulatory side effects caused by PDE V inhibitors, in particular during simultaneous administration of vasodilators which act via the NO-cGMP system.

Preference is given to vasodilators selected from the group consisting of the nitrate compounds.

The invention also relates to the use of a highly penis-specific PDE V subtype inhibitor, or a physiologically acceptable salt and/or solvate thereof, for the preparation of a medicament for the treatment of sexual disorders in women without circulatory side effects caused by PDE V inhibitors. Preference is given here to the use of a highly penis-specific PDE V inhibitor, or a physiologically acceptable salt and/or solvate thereof, for the preparation of a medicament for the treatment of sexual disorders in women without circulatory side effects caused by PDE V inhibitors.

The invention preferably relates to the use as described above, where the PDE V inhibitor is a compound of the formula I below

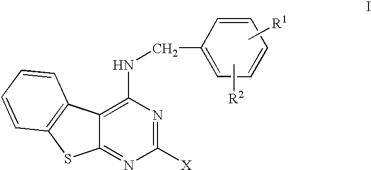

in which

R¹ and R² are each, independently of one another, H, A, OA, OH or Hal,

R¹ and R² together are alternatively alkylene having 3-5 carbon atoms, —O—CH₂—CH₂—, —CH₂—O—CH₂—, —O—CH₂—O— or —O—CH₂—CH₂—O—,

X is R⁴, R⁵ or R⁶, each of which is monosubstituted by R⁷,

R⁴ is linear or branched alkylene having 1-10 carbon atoms, in which one or two CH₂ groups may be replaced by —CH═CH— groups,

R⁵ is cycloalkyl or cycloalkylalkylene having 5-12 carbon atoms,

R⁶ is phenyl or phenylmethyl,

R⁷ is COOH, COOA, CONH₂, CONHA, CON(A)₂ or CN,

A is alkyl having 1 to 6 carbon atoms, and

Hal is F, Cl, Br or I,

and physiologically acceptable salts and/or solvates thereof.

The invention relates in particular to the use as described above, where the PDE V inhibitor is selected from the group consisting of

(a) 3-[4-(3-chloro-4-methoxybenzylamino)benzo[4,5]thieno[2,3-d]-pyrimidin-2-yl]propionic acid;

(b) 4-[4-(3,4-methylenedioxybenzylamino)benzo[4,5]thieno[2,3-d]-pyrimidin-2-yl]butyric acid;

(c) 7-[4-(3,4-methylenedioxybenzylamino)benzo[4,5]thieno[2,3-d]-pyrimidin-2-yl]heptanoic acid;

(d) 7-[4-(3-chloro-4-methoxybenzylamino)benzo[4,5]thieno[2,3-d]-pyrimidin-2-yl]heptanoic acid;

(e) 5-[4-(3-chloro-4-methoxybenzylamino)benzo[4,5]thieno[2,3-d]-pyrimidin-2-yl]valeric acid;

(f) 2-{4-[4-(3-chloro-4-methoxybenzylamino)benzo[4,5]thieno[2,3-d]-pyrimidin-2-yl]cyclohexyl-1-yl}acetic acid;

(g) 4-[4-(3 ,4-methylened ioxybenzylamino)benzo[4,5]thieno[2 ,3-d]-pyrimidin-2-yl]cyclohexanecarboxylic acid;

(h) 4-[4-(3,4-methylenedioxybenzylamino)benzo[4,5]thieno[2,3-d]-pyrimidin-2-yl]benzoic acid;

(i) 4-[4-(3,4-methylenedioxybenzylamino)benzo[4,5]thieno[2,3-d]-pyrimidin-2-yl]phenylacetic acid;

(k) 4-[4-(3-chloro-4-methoxybenzylamino)benzo[4,5]thieno[2,3-d]-pyrimidin-2-yl]cyclohexanecarboxylic acid, ethanolamine salt

and physiologically acceptable salts and/or solvates thereof.

The invention furthermore relates to the compounds of the formula I

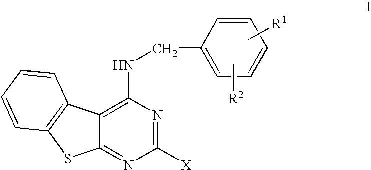

in which

R¹ and R² are each, independently of one another, H, A, OA, OH or Hal,

R¹ and R² together are alternatively alkylene having 3-5 carbon atoms, —O—CH₂—CH₂—, —CH₂—O—CH₂—, —O—CH₂—O— or —O—CH₂—CH₂—O—,

X is R⁴, R⁵ or R⁶, each of which is monosubstituted by R⁷,

R⁴ is linear or branched alkylene having 1-10 carbon atoms, in which one or two CH₂ groups may be replaced by —CH═CH— groups,

R⁵ is cycloalkyl or cycloalkylalkylene having 5-12 carbon atoms,

R⁶ is phenyl or phenylmethyl,

R⁷ is COOH, COOA, CONH₂, CONHA, CON(A)₂ or CN,

A is alkyl having 1 to 6 carbon atoms, and

Hal is F, Cl, Br or I,

and physiologically acceptable salts and/or solvates thereof, as highly penis-specific PDE V inhibitors.

In the compounds of the formula I described, the selective action takes place specifically at the penis

a) by inhibition of a penis-specific sub-type of phosphodiesterase V, and/or

b) as a consequence of selective transport into the penis effector cell or by rapid elimination from the effector cells of the cardiovascular system.

The compounds of the formula I can be employed as medicament active ingredients in human and veterinary medicine. They can furthermore be employed as intermediates for the preparation of further medicament active ingredients.

Above and below, the radicals R¹, R², R³, R⁴, R⁵, R⁶, R⁷, X and L are as defined under the formulae I, II and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁴, R⁵ or R⁶ radical which is monosubstituted by R⁷.

R⁴ is a linear or branched alkylene radical having 1-10 carbon atoms, where the alkylene radical is preferably, for example, methylene, ethylene, propylene, isopropylene, butylene, isobutylene, sec-butylene, pentylene, 1-, 2- or 3-methylbutylene, 1,1-, 1,2- or 2,2-dimethylpropylene, 1-ethylpropylene, hexylene, 1-, 2-, 3- or 4-methylpentylene, 1,1-, 1,2-, 1,3-, 2,2-, 2,3- or 3,3-dimethylbutylene, 1- or 2-ethylbutylene, 1-ethyl-1-methylpropylene, 1-ethyl-2-methylpropylene, 1,1,2- or 1,2,2-trimethylpropylene, linear or branched heptylene, octylene, nonylene or decylene.

R⁵ is furthermore, for example, but-2-enylene or hex-3-enylene.

Very particular preference is given to ethylene, propylene and butylene. R⁵ is cycloalkylalkylene having 5-12 carbon atoms, preferably, for example, cyclopentylmethylene, cyclohexylmethylene, cyclohexylethylene, cyclohexylpropylene or cyclohexylbutylene.

R⁵ is also cycloalkyl, preferably having 5-7 carbon atoms. Cycloalkyl is, for example, cyclopentyl, cyclohexyl or cycloheptyl.

Hal is preferably F, Cl or Br, but also I.

The radicals R¹ and R² may be identical or different and are preferably in the 3- or 4-position of the phenyl ring. They are each, for example, independently of one another, H, alkyl, F, Cl, Br or I or together are alkylene, such as, for example, propylene, butylene or pentylene, furthermore ethyleneoxy, methylenedioxy or ethylenedioxy. They are preferably also each alkoxy, such as, for example, methoxy, ethoxy or propoxy, furthermore hydroxyl.

The radical R⁷ is preferably, for example, COOH, COOCH₃, COOC₂H₅, CONH₂, CON(CH₃)₂, CONHCH₃ or CN.

Throughout the invention, all radicals which occur more than once may be identical or different, i.e. are independent of one another.

Accordingly, the invention relates in particular to the use of the compounds of the formula I in which at least one of the said radicals has one of the preferred meanings indicated above. Some preferred groups of compounds may be expressed by the following sub-formulae Ia to Id, which conform to the formula I and in which the radicals not designated in greater detail are as defined under the formula I, but in which

in Ia X is R⁴ which is substituted by COOH, COOA, CONH₂, CONA₂, CONHA or CN, or is phenyl or phenylmethyl;

in Ib R¹ and R² together are alkylene having 3-5 carbon atoms, —O—CH₂—CH₂—, —O—CH₂—O— or —O—CH₂—CH₂—O—,

X is R⁴ which is substituted by COOH, COOA, CONH₂, CONA₂, CONHA or CN, or is phenyl or phenylmethyl;

in Ic R¹ and R² are each, independently of one another, H, A, OA or Hal,

R¹ and R² together are alkylene having 3-5 carbon atoms, —O—CH₂—CH₂—, —O—CH₂—O— or —O—CH₂—CH₂—O—,

X is R⁴ which is substituted by COOH, COOA, CONH₂, CONA₂, CONHA or CN, or is phenyl or phenylmethyl;

in Id R¹ and R² are each, independently of one another, H, A, OA or Hal,

R¹ and R² together are alternatively alkylene having 3-5 carbon atoms, —O—CH₂—CH₂—, —O—CH₂—O— or —O—CH₂—CH₂—O—,

X is alkylene having 2-5 carbon atoms which is mono-substituted by R⁷, or is cyclohexyl, phenyl or phenylmethyl,

R⁷ is COOH or COOA,

A is alkyl having 1 to 6 carbon atoms,

Hal is F, Cl, Br or I.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuftgart), to be precise under reaction conditions which are known and suitable for said reactions. Use can also be made here of variants which are known per se, but are not mentioned here in greater detail.

In the compounds of the formula II or III, R¹, R², R³, R⁴, X and n have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, furthermore also 2-naphthalenesulfonyloxy).

The compounds of the formula I can preferably be obtained by reacting compounds of the formula II with compounds of the formula III.

If desired, the starting materials can also be formed in situ by not isolating them from the reaction mixture, but instead immediately converting them further into the compounds of the formula I.

On the other hand, it is possible to carry out the reaction stepwise.

The starting compounds of the formula II and III are generally known. If they are not known, they can be prepared by methods known per se. Compounds of the formula II can be obtained, for example, from the corresponding hydroxypyrimidines, which are built up from thiophene derivatives and CN-substituted alkylenecarboxylic acid esters (Eur. J. Med. Chem. 23, 453 (1988)), by reaction with POCl₃.

The hydroxypyrimidines are prepared either by dehydrogenation of corresponding tetrahydrobenzothienopyrimidine compounds or by the cyclisation of 2-aminobenzothiophene-3-carboxylic acid derivatives using aldehydes or nitriles which is conventional for the preparation of pyrimidine derivatives (for example Houben-Weyl E9b/2).

In detail, the reaction of the compounds of the formula II with the compounds of the formula III is carried out in the presence or absence of an inert solvent at temperatures between about −20 and about 150°, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of said solvents.

It is furthermore possible to convert a radical X in a compound of the formula I into another radical X, for example by hydrolysing an ester or a cyano group to give a COOH group.

Ester groups can be saponified, for example using NaOH or KOH in water, water/THF or water/dioxane at temperatures between 0 and 100°. Carboxylic acids can be converted into the corresponding carboxylic acid chlorides, for example using thionyl chloride, and these can be converted into carboxamides. Elimination of water therefrom in a known manner gives carbonitriles.

An acid of the formula I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I can be converted into the corresponding metal salt, in particular alkali metal or alkaline earth metal salt, or into the corresponding ammonium salt, using a base (for example sodium hydroxide, potassium hydroxide, sodium carbonate or potassium carbonate). Also suitable for this reaction are, in particular, organic bases which give physiologically acceptable salts, such as, for example, ethanolamine.

On the other hand, a base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

The invention furthermore relates to pharmaceutical preparations comprising at least one highly penis-specific PDE V inhibitor, or a physiologically acceptable salt and/or solvate thereof, for the treatment of erectile dysfunction in men without circulatory side effects caused by PDE V inhibitors.

In the pharmaceutical preparations, preference is given to the compounds of the formula I.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilized and the resultant lyophilizates used, for example, for the preparation of injection preparations. The preparations indicated may be sterilized and/or comprise assistants, such as lubricants, preservatives, stabilizers and/or wetting agents, emulsifiers, salts for modifying the osmotic pressure, buffer substances, colorants and flavours and/or a plurality of further active ingredients, for example one or more vitamins.

The compounds of the formula I and their physiologically acceptable salts can be employed for combating illnesses in which an increase in the cGMP (cycloguanosine monophosphate) level results in inflammation inhibition or reduction and muscle relaxation. The compounds according to the invention are used in particular in the treatment of angina, high blood pressure, high pulmonary pressure, congestive heart failure, atherosclerosis, conditions involving reduced passage through heart vessels, peripheral vascular diseases, strokes, bronchitis, allergic asthma, chronic asthma, allergic rhinitis, glaucoma, irritable bowel syndrome, tumours, kidney insufficiency, liver cirrhosis and for the treatment of female impotence.

In general, the substances are preferably administered in doses of from about 1 to 500 mg, in particular from 5 to 100 mg, per dosage unit. The daily dose is preferably from about 0.02 to 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Pharmacological Test Report

Investigations of trans-4-[4-(3-chloro-4-methoxybenzylamino)benzo[4,5]-thieno[2,3-d]pyrimidin-2-yl]cyclohexanecarboxylic acid, ethanolamine salt (EMD 221829) for haemodynamics and for erectile function in various animal species, in some cases with simultaneous administration of nitrate, compared with sildenafil citrate.

All dose amounts are i.d. (intraduodenal), unless stated otherwise.

A

Measurements of Erectile Function in Anaesthetized Dogs with Closed Thorax

Erections of various extent were initiated by stimulation variation of the nervus pelvinus. The pressure in the corpus cavernosum rigidum [CCR] was measured.

Result:

EMD 221829 stimulates submaximal erection at only 1 mg of substance/1 kg of body weight.

Corresponding results are measured for sildenafil.

At the same time, the blood pressure and heart rate were measured.

In contrast to sildenafil, no effect on these haemodynamic parameters is evident at 1 mg/kg in the case of EMD 221829.

B

Extensive studies on the haemodynamics of EMD 221829 on anaesthetized dogs with open thorax showed no cardiovascular side effects, such as, for example, a reduction in blood pressure with a reflex increase in heart rate.

At 1 mg of substance/1 kg of body weight, a clear difference in the haemodynamic parameters compared with sildenafil is measured.

In the case of EMD 221829, effects on the blood pressure and heart rate are only measurable from 100 mg of substance/1 kg of body weight. In this dose, sildenafil citrate causes a reduction in blood pressure and an increase in heart rate.

C

Investigations of Nitrate Interaction of EMD 221829 and Sildenafil

The model and administration are as in B, but both test substances are administered with simultaneous permanent i.v. infusion of isosorbitol dinitrate (50 μg/kg of body weight/hour).

Result:

The sildenafil-induced changes in the above-mentioned parameters are significantly increased by simultaneous administration of nitrate. By contrast, no changes in the initial parameters are evident for EMD 221829 under the same conditions.

D

Telemetric Measurements of Blood Pressure and Heart Rate Over 24 Hours in Spontaneously Hypertensive Rats

Measurements of 3, 10 and 30 mg of substance/1 kg of body weight/p.o.

After administration of sildenafil, a significant dose-dependent reduction in blood pressure occurs. In the case of EMD 221829, no change in blood pressure is evident in the same dose range.

E

as for D in Normotensive Wistar Rats

Measurements of 30, 100 and 300 mg of substance/1 kg of body weight/p.o.

Result:

After administration of sildenafil, a significant dose-dependent reduction in blood pressure and an increase in hear rate occur.

On administration of EMD 221829, no changes are evident.

F

Nitrate Interaction in Spontaneously Hypertensive Rats

Method: surgical instrumentation and measurement of blood pressure and heart rate after intravenous cumulative administration of either EMD 221829 or sildenafil (0.1, 0.3, 1, 3 or 10 mg/kg of body weight) with simultaneous infusion of isosorbitol dinitrate (100 μg/kg of body weight/hour).

Result: a dose-dependent drop in blood pressure and increase in heart rate are observed in the case of sildenafil, whereas no change is observed in the case of EMD 221829.

### CONCLUSION

The pharmacological studies confirm the efficacy of highly penis-specific PDE V inhibitors, which enable treatment of erectile dysfunction in men and sexual disorders in women without adversely affecting the cardiovascular system, in particular with simultaneous nitrate treatment.

Preparation of the Compounds

Above and below, all temperatures are given in ° C. In the examples below, “conventional work-up” means that water is added if necessary, a pH of from 2 to 10, depending on the constitution of the end product, is set if necessary, the mixture is extracted with ethyl acetate or dichloromethane, the phases are separated, the organic phase is dried over sodium sulfate and evaporated, and the product is purified by chromatography on silica gel and/or by crystallization.

Mass spectrometry (MS): EI (electron impact ionisation) M⁺ FAB (fast atom bombardment) (M+H)⁺

### EXAMPLE 1

Methyl 3-(4-chlorobenzothieno[2, 3-d]pyrimid in-2-yl)propionate [obtainable by cyclisation of methyl 2-amino-5,6,7,8-tetrahydrobenzothiophene-3-carboxylate using methyl 3-cyanopropionate, dehydrogenation using sulfur and subsequent chlorination using phosphorus oxychloride/dimethylamine] and 3-chloro-4-methoxybenzylamine (“A”) in N-methylpyrrolidone are stirred at 110° for 5 hours. The solvent is removed, and the product is subjected to conventional work-up, giving methyl 3-[4-(3-chloro-4-methoxybenzylamino)benzothieno[2,3-d]pyrimidin-2-yl]propionate as a colourless oil.

Analogous Reaction of “A”

with methyl 2-(4-chlorobenzothieno[2,3-d]pyrimidin-2-yl)acetate gives

methyl 2-(4-(3-chloro-4-methoxybenzylamino)benzothieno[2,3-d]-pyrimidin-2-yl)acetate.

Analogous Reaction of 3,4-methylenedioxybenzylamine

with methyl 3-(4-chlorobenzothieno[2,3-d]pyrimidin-2-yl)propionate gives

methyl 3-[4-(3,4-methylenedioxybenzylamino)benzothieno[2,3-d]-pyrimidin-2-yl]propionate.

Analogous Reaction of “A”

with methyl 4-(4-chlorobenzothieno[2,3-d]pyrimidin-2-yl)butyrate gives

methyl 4-[4-(3-chloro-4-methoxybenzylamino)benzothieno[2,3-d]-pyrimidin-2-yl)butyrate.

Analogous Reaction of 3,4-methylenedioxybenzylamine

with methyl 4-(4-chlorobenzothieno[2,3-d]pyrimidin-2-yl)butyrate gives

methyl 4-[4-(3,4-methylenedioxybenzylamino)benzothieno[2,3-d]-pyrimidin-2-yl)butyrate.

Analogous Reaction of “A”

with methyl 5-(4-chlorobenzothieno[2,3-d]pyrimidin-2-yl)valerate gives

methyl 5-[4-(3-chloro-4-methoxybenzylamino)benzothieno[2,3-d]-pyrimidin-2-yl)valerate

Analogous Reaction of 3,4-methylenedioxybenzylamine

with methyl 5-(4-chlorobenzothieno[2 ,3-d]pyrimidin-2-yl)valerate gives

methyl 5-[4-(3,4-methylenedioxybenzylamino)benzothieno[2,3-d]-pyrimidin-2-yl)valerate.

Analogous Reaction of “A”

with methyl 7-(4-chlorobenzothieno[2,3-d]pyrimidin-2-yl)heptanoate gives

methyl 7-[4-(3-chloro-4-methoxybenzylamino)benzothieno[2,3-d]-pyrimidin-2-yl)heptanoate

Analogous Reaction of 3,4-methylenedioxybenzyiamine

with methyl 7-(4-chlorobenzothieno[2,3-d]pyrimidin-2-yl)heptanoate gives

methyl 7-[4-(3,4-methylened ioxybenzylamino)benzothieno[2, 3-d]-pyrimidin-2-yl)heptanoate.

Analogous Reaction of “A”

with methyl 2-[4-(4-chlorobenzothieno[2,3-d]pyrimidin-2-yl)cyclohex-1-yl]-acetate gives

methyl 2-{4-[4-(3-chloro-4-methoxybenzylamino)benzothieno[2,3-d]-pyrimidin-2-yl)cyclohexyl-1-yl}acetate.

Analogous Reaction of 3,4-methylenedioxybenzylamine

with methyl 2-[4-(4-chlorobenzothieno[2 ,3-d]pyrimidin-2-yl)cyclohex-1-yl]-acetate gives

methyl 2-{4-[4-(3,4-methylenedioxybenzylamino)benzothieno[2,3-d]-pyrimidin-2-yl)cyclohexyl-1-yl}acetate.

Analogous Reaction of Benzylamine

with methyl 3-(4-chlorobenzothieno[2,3-d]pyrimidin-2-yl)propionate gives

methyl 3-(4-benzylaminobenzothieno[2,3-d]pyrimidin-2-yl)-propionate;

with methyl 4-(4-chlorobenzothieno[2,3-d]pyrimidin-2-yl)butyrate gives

methyl 4-(4-benzylaminobenzothieno[2,3-d]pyrimidin-2-yl)butyrate;

with methyl 5-(4-chorobenzothieno[2,3-d]pyrimidin-2-yl)valerate gives

methyl 5-(4-benzylaminobenzothieno[2,3-d]pyrimidin-2-yl)valerate

Analogous Reaction of “A”

with methyl 4-(4-chlorobenzothieno[2,3-d]pyrimidin-2-yl)cyclohexane-carboxylate gives

methyl 4-[4-(3-chloro-4-methoxybenzylamino)benzothieno[2,3-d]-pyrimidin-2-yl)cyclohexanecarboxylate

and reaction of 3,4-methylenedioxybenzylamine gives

methyl 4-[4-(3,4-methylened ioxybenzylamino)benzothieno[2,3-d]-pyrimidin-2-yl)cyclohexanecarboxylate.

### EXAMPLE 2

Methyl 3-[4-(3-chloro-4-methoxybenzylamino)benzothieno[2,3-d]pyrimidin-2-yl)propionate is dissolved in ethylene glycol monomethyl ether, 32% NaOH is added, and the mixture is stirred at 110° for 5 hours. 20% HCl is added, and the mixture is extracted with dichloromethane. Addition of petroleum ether gives 3-[4-(3-chloro-4-methoxybenzylamino)benzothieno-[2,3-d]pyrimidin-2-yl]propionic acid, m.p. 218°.

The precipitated crystals are dissolved in isopropanol, and ethanolamine is added. Crystallization gives 3-[4-(3-chloro-4-methoxybenzylamino)benzothieno[2,3-d]pyrimidin-2-yl]propionic acid, ethanolamine salt.

The following compounds are obtained analogously:

4-[4-(3-chloro-4-methoxybenzylamino)benzothieno[2,3-d]pyrimidin-2-yl]butyric acid, m.p. 225°; ethanolamine salt m.p. 150°;

5-[4-(3-chloro-4-methoxybenzylamino)benzothieno[2 ,3-d]pyrimidin-2-yl]valeric acid, m.p. 210°; ethanolamine salt m.p. 141°;

4-[4-(3,4-methylenedioxybenzylamino)benzothieno[2,3-d]pyrimidin-2-yl]butyric acid, hydrochloride, m.p. 245°.

The following carboxylic acids are obtained analogously from the esters listed under Example 1:

2-[4-(3-chloro-4-methoxybenzylamino)benzothieno[2 ,3-d]pyrimidin-2-yl]acetic acid,

3-[4-(3,4-methylenedioxybenzylamino)benzothieno[2,3-d]pyrimidin-2-yl]propionic acid,

5-[4-(3,4-methylenedioxybenzylamino)benzothieno[2,3-d]pyrimidin-2-yl]valeric acid,

7-[4-(3-chloro-4-methoxybenzylamino)benzothieno[2,3-d]pyrimidin-2-yl]heptanoic acid,

7-[4-(3 ,4-methylenedioxybenzylamino)benzothieno[2,3-d]pyrimidin-2-yl]heptanoic acid,

2-{4-[4-(3-chloro-4-methoxybenzylamino)benzothieno[2 ,3-d]-pyrimidin-2-yl]cyclohexyl-1-yl}acetic acid,

2-{4-[4-(3,4-methylenedioxybenzylamino)benzothieno[2,3-d]-pyrimidin-2-yl]cyclohexyl-1-yl}acetic acid,

3-(4-benzylaminobenzothieno[2,3-d]pyrimidin-2-yl]propionic acid,

4-(4-benzylaminobenzothieno[2,3-d]pyrimidin-2-yl]butyric acid,

5-(4-benzylaminobenzothieno[2,3-d]pyrimidin-2-yl]valeric acid,

4-[4-(3-chloro-4-methoxybenzylamino)benzothieno[2,3-d]pyrimidin-2-yl]cyclohexanecarboxylic acid, ethanolamine salt, m.p. 167°;

4-[4-(3 ,4-methylened ioxybenzylamino)benzothieno[2,3-d]pyrimidin-2-yl]cyclohexanecarboxylic acid, ethanolamine salt, m.p. 143°.

### EXAMPLE 3

A mixture of 1.5 g of methyl 4-(4-chlorobenzothieno[2,3-d]pyrimidin-2-yl)-phenylcarboxylate (“B”), prepared by dehydrogenation of the corresponding 5,6,7,8-tetrahydrobenzothieno[2,3-d]pyrimidine compound using sulfur followed by chlorination using phosphorus oxychloride/dimethylamine, and 1.5 g of 3-chloro-4-methoxybenzylamine in 20 ml of N-methylpyrrolidone is warmed at 110° C. for 4 hours. After cooling, the mixture is subjected to conventional work-up, giving 2.6 g of methyl 4-[4-(3-chloro-4-methoxybenzylamino)-[1]benzothieno[2,3-d]pyrimidin-2-yl]benzoate, m .p. 203-204°.

Analogously to Example 2, 1.2 g of the ester give 1.0 g of

4-[4-(3-chloro-4-methoxybenzylamino)-[1]benzothieno[2,3-d]-pyrimidin-2-yl]benzoic acid, ethanolamine salt, m.p. 189-190°.

Analogously to Example 1, “B” and 3,4-methylenedioxybenzylamine give

methyl 4-[4-(3,4-methylenedioxybenzylamino)-[1]benzothieno[2,3-d]-pyrimidin-2-yl]benzoate, and ester hydrolysis thereof gives 4-[4-(3,4-methylenedioxybenzylamino)-[1]benzothieno[2,3-d]-pyrimidin-2-yl]benzoic acid, sodium salt, m.p. >260°.

The compounds

4-[4-(3-chloro-4-methoxybenzylamino)-[1]benzothieno[2,3-d]-pyrimidin-2-yl]phenylacetic acid, ethanolamine salt, m.p. 130°; and

4-[4-(3,4-methylenedioxybenzylamino)-[1]benzothieno[2,3-d]-pyrimidin-2-yl]phenylacetic acid, ethanolamine salt, m.p. 202° are obtained analogously.

### EXAMPLE 4

1 equivalent of 3-[4-(3-chloro-4-methoxybenzylamino)benzothieno[2 ,3-d]-pyrimidin-2-yl]propionic acid and 1.2 equivalents of thionyl chloride are stirred in dichloromethane for 2 hours. The solvent is removed, giving 3-[4-(3-chloro-4-methoxybenzylamino)benzothieno[2,3-d]pyrimidin-2-yl]-propionyl chloride.

The product is transferred into aqueous ammonia, and the mixture is stirred for one hour and subjected to conventional work-up, giving 3-[4-(3-chloro-4-methoxybenzylamino)benzothieno[2,3-d]pyrimidin-2-yl]propionamide.

### EXAMPLE 5

1 equivalent of DMF and 1 equivalent of oxalyl chloride are dissolved in acetonitrile at 0°. 1 equivalent of 3-[4-(3-chloro-4-methoxybenzylamino)-benzothieno[2,3-d]pyrimidin-2-yl]propionamide is then added. The mixture is stirred for a further one hour. Conventional work-up gives 3-[4-(3-chloro-4-methoxybenzylamino)benzothieno[2,3-d]pyrimidin-2-yl]propionitrile.

### EXAMPLE 6

The following carboxylic acids are obtained analogously to Examples 1, 2 and 3 by reaction of the corresponding chloropyrimidine derivatives with 3,4-ethylenedioxybenzylamine:

4-[4-(3,4-ethylenedioxybenzylamino)benzothieno[2,3-d]pyrimidin-2-yl]butyric acid,

3-[4-(3,4-ethylenedioxybenzylamino)benzothieno[2,3-d]pyrimidin-2-yl]propionic acid,

5-[4-(3,4-ethylened ioxybenzylamino)benzothieno[2,3-d]pyrimidin-2-yl]valeric acid,

7-[4-(3,4-ethylenedioxybenzylamino)benzothieno[2,3-d]pyrimidin-2-yl]heptanoic acid,

2-{4-[4-(3,4-ethylenedioxybenzylamino)benzothieno(2,3-d]pyrimidin-2-yl]cyclohexyl-1-yl}acetic acid,

4-[4-(3,4-ethylenedioxybenzylamino)benzothieno[2,3-d]pyrimidin-2-yl]cyclohexanecarboxylic acid,

4-[4-(3,4-ethylenedioxybenzylamino)-[1]benzothieno[2,3-d]pyrimidin-2-yl]benzoic acid, decomp. 220-230°;

4-[4-(3 ,4-ethylened ioxybenzylamino)-[1]benzothieno[2,3-d]pyrimidin-2-yl]benzoic acid, ethanolamine salt, m.p. 252°;

4-[4-(3,4-ethylenedioxybenzylamino)-[1]benzothieno[2,3-d]pyrimidin-2-yl]phenylacetic acid.

The following compounds are obtained analogously by reaction with 3,4-dichlorobenzylamine:

4-[4-(3,4-d ichlorobenzylamino)benzothieno[2,3-d]pyrimid in-2-yl]-butyric acid,

3-[4-(3,4-dichlorobenzylamino)benzothieno[2,3-d]pyrimidin-2-yl]-propionic acid,

5-[4-(3 ,4-dichlorobenzylamino)benzothieno[2,3-d]pyrimidin-2-yl]-valeric acid, ethanolamine salt, m.p. 160°;

7-[4-(3 ,4-dichlorobenzylamino)benzothieno[2,3-d]pyrimidin-2-yl]-heptanoic acid,

2-{4-[4-(3,4-dichlorobenzylamino)benzothieno[2,3-d]pyrimidin-2-yl]-cyclohexyl-1-yl}acetic acid,

4-[4-(3 ,4-d ichlorobenzylamino)benzothieno[2 ,3-d]pyrimidin-2-yl]-cyclohexanecarboxylic acid,

4-[4-(3,4-dichlorobenzylamino)-[1]benzothieno[2,3-d]pyrimidin-2-yl]-benzoic acid,

4-[4-(3,4-dichlorobenzylamino)-[1]benzothieno[2,3-d]pyrimidin-2-yl]-phenylacetic acid.

The following compounds are obtained analogously by reaction with 3-chloro-4-ethoxybenzylamine:

4-[4-(3-chloro-4-ethoxybenzylamino)benzothieno[2,3-d]pyrimidin-2-yl]butyric acid,

3-[4-(3-chloro-4-ethoxybenzylamino)benzothieno[2,3-d]pyrimidin-2-yl]propionic acid,

5-[4-(3-chloro-4-ethoxybenzylamino)benzothieno[2,3-d]pyrimidin-2-yl]valeric acid,

7-[4-(3-chloro-4-ethoxybenzylamino)benzothieno[2,3-d]pyrimidin-2-yl]heptanoic acid,

2-{4-[4-(3-chloro-4-ethoxybenzylamino)benzothieno[2,3-d]pyrimidin-2-yl]cyclohexyl-1-yl}acetic acid,

4-[4-(3-chloro-4-ethoxybenzylamino)benzothieno[2, 3-d]pyrimidin-2-yl]cyclohexanecarboxylic acid,

4-[4-(3-chloro-4-ethoxybenzylamino)-[1]benzothieno[2,3-d]pyrimidin-2-yl]benzoic acid, m.p. 185-187°;

4-[4-(³-chloro-4-ethoxybenzylamino)-[1]benzothieno[2,3-d]pyrimidin-2-yl]phenylacetic acid.

The following compounds are obtained analogously by reaction with 3-chloro-4-isopropoxybenzylamine:

4-[4-(3-chloro-4-isopropoxybenzylamino)benzothieno[2,3-d]-pyrimidin-2-yl]butyric acid,

3-[4-(3-chloro-4-isopropoxybenzylamino)benzothieno[2,3-d]-pyrimidin-2-yl]propionic acid,

5-[4-(3-chloro-4-isopropoxybenzylamino)benzothieno[2,3-d]-pyrimidin-2-yl]valeric acid, ethanolamine salt, m.p. 130°;

7-[4-(3-chloro-4-isopropoxybenzylamino)benzothieno[2,3-d]-pyrimidin-2-yl]heptanoic acid,

2-{4-[4-(3-chloro-4-isopropoxybenzylamino)benzothieno[2,3-d]-pyrimidin-2-yl]cyclohexyl-1-yl}acetic acid,

4-[4-(3-chloro-4-isopropoxybenzylamino)benzothieno[2,3-d]-pyrimidin-2-yl]cyclohexanecarboxylic acid,

4-[4-(3-chloro-4-isopropoxybenzylamino)-[1]benzothieno[2,3-d]-pyrimidin-2-yl]benzoic acid, m.p. 240-241°;

4-[4-(3-chloro-4-isopropoxybenzylamino)-[1]benzothieno[2,3-d]-pyrimidin-2-yl]phenylacetic acid.

The examples below relate to pharmaceutical preparations:

### EXAMPLE A

### Injection Vials

A solution of 100 g of an active ingredient of the formula 1 and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilized by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of an active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of an active ingredient of the formula I are introduced into hard gelatine capsules in a conventional manner in such a way that each capsule contains 20 mg of the active ingredient.

### EXAMPLE H

### Ampoules

A solution of 1 kg of an active ingredient of the formula I in 60 l of bidistilled water is sterile filtered, transferred into ampoules, lyophilised under sterile conditions and sealed under sterile conditions. Each ampoule contains 10 mg of active ingredient.

### EXAMPLE I

### Inhalation Spray

14 g of an active ingredient of the formula I are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg.

1. Use of a highly penis-specific PDE V inhibitor, or a physiologically acceptable salt and/or solvate thereof, for the preparation of a medicament for the treatment of erectile dysfunction in men without circulatory side effects caused by PDE V inhibitors.

2. Use according to claim 1 for the preparation of a medicament for the treatment of erectile dysfunction in men without circulatory side effects caused by PDE V inhibitors, in particular during simultaneous administration of vasodilators which act via the NO-cGMP system.

3. Use according to claim 3, where the vasodilators are selected from the group consisting of nitrate compounds.

4. Use according to one of the preceding claims, where the PDE V inhibitor is a compound of the formula I 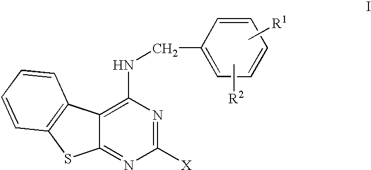
in which
R¹ and R² are each, independently of one another, H, A, OA, OH or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
X is R⁴, R⁵ or R⁶, each of which is monosubstituted by R⁷,
R⁴ is linear or branched alkylene having 1-10 carbon atoms, in which one or two CH₂ groups may be replaced by —CH═CH— groups,
R⁵ is cycloalkyl or cycloalkylalkylene having 5-12 carbon atoms,
R⁶ is phenyl or phenylmethyl,
R⁷ is COOH, COOA, CONH₂, CONHA, CON(A)₂ or CN,
A is alkyl having 1 to 6 carbon atoms, and
Hal is F, Cl, Br or I.

5. Use according to one of the preceding claims, where the PDE V inhibitor is selected from the group consisting of
(a) 3-[4-(3-chloro-4-methoxybenzylamino)benzo[4,5]thieno-[2,3-d]pyrimidin-2-yl]propionic acid;
(b) 4-[4-(3,4-methylenedioxybenzylamino)benzo[4,5]thieno-[2,3-d]pyrimidin-2-yl]butyric acid;
(c) 7-[4-(3,4-methylenedioxybenzylamino)benzo[4,5]thieno-[2,3-d]pyrimidin-2-yl]heptanoic acid;
(d) 7-[4-(3-chloro-4-methoxybenzylamino)benzo[4,5]thieno-[2,3-d]pyrimidin-2-yl]heptanoic acid;
(e) 5-[4-(3-chloro-4-methoxybenzylamino)benzo[4,5]thieno-[2,3-d]pyrimidin-2-yl]valeric acid;
(f) 2-{4-[4-(3-chloro-4-methoxybenzylamino)benzo[4,5]thieno-[2,3-d]pyrimidin-2-yl]acetic acid;
(g) 4-[4-(3,4-methylenedioxybenzylamino)benzo[4,5]thieno-[2,3-d]pyrimid in-2-yl]cyclohexanecarboxylic acid;
(h) 4-[4-(3,4-methylenedioxybenzylamino)benzo[4,5]thieno-[2,3-d]pyrimidin-2-yl]benzoic acid;
(i) 4-[4-(3,4-methylenedioxybenzylamino)benzo[4,5]thieno-[2,3-d]pyrimid in-2-yl]phenylacetic acid;
(k) 4-[4-(3-chloro-4-methoxybenzylamino)benzo[4,5]thieno-[2,3-d]pyrimidin-2-yl]cyclohexanecarboxylic acid, ethanolamine salt;
and physiologically acceptable salts and/or solvates thereof.

6. Compounds of the formula I 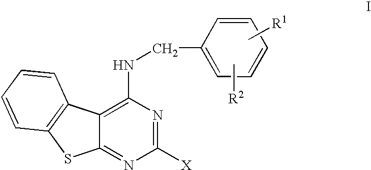
in which
R¹ and R² are each, independently of one another, H, A, OA, OH or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
X is R⁴, R⁵ or R⁶, each of which is monosubstituted by R⁷,
R⁴ is linear or branched alkylene having 1-10 carbon atoms, in which one or two CH₂ groups may be replaced by —CH═CH— groups,
R⁵ is cycloalkyl or cycloalkylalkylene having 5-12 carbon atoms,
R⁶ is phenyl or phenylmethyl,
R⁷ is COOH, COOA, CONH₂, CONHA, CON(A)₂ or CN,
A is alkyl having 1 to 6 carbon atoms, and
Hal is F, Cl, Br or I,
and physiologically acceptable salts and/or solvates thereof, as highly penis-specific PDE V inhibitors.

7. Pharmaceutical preparation comprising at least one highly penis-specific PDE V inhibitor, or a physiologically acceptable salt and/or solvate thereof, for the treatment of erectile dysfunction in men without circulatory side effects caused by PDE V inhibitors.

8. Pharmaceutical preparation according to claim 7, comprising at least one highly penis-specific PDE V inhibitor of the formula I according to claim 6, or a physiologically acceptable salt and/or solvate thereof, for the treatment of erectile dysfunction in men without circulatory side effects caused by PDE V inhibitors.
